(19) United States
(12) Patent Application Publication
(10) Pub. No.: US 2022/0016187 A1
(43) Pub. Date: Jan. 20, 2022

(54) LACTOBACILLUS PARACASEI AND ITS APPLICATION IN PREPARATION OF MEDICINE FOR TREATING ULCERATIVE COLITIS

(71) Applicants: Foshan Langsee Biotechnology Co. Ltd., Foshan (CN); Guangdong Longsee Biomedical Co. Ltd., Guangzhou (CN)

(72) Inventors: Jincheng ZENG, Guangzhou (CN); Juan HUANG, Guangzhou (CN); Weiqing YANG, Guangzhou (CN); Zhao ZHANG, Guangzhou (CN); Ziyan YANG, Guangzhou (CN); Bihua LIN, Guangzhou (CN); Tao CHEN, Guangzhou (CN); Chunyan LEI, Guangzhou (CN)

(73) Assignees: Foshan Langsee Biotechnology Co. Ltd., Foshan (CN); Guangdong Longsee Biomedical Co. Ltd., Guangzhou (CN)

(21) Appl. No.: 17/130,146

(22) Filed: Dec. 22, 2020

Related U.S. Application Data
(63) Continuation of application No. PCT/CN2020/128122, filed on Nov. 11, 2020.

(30) Foreign Application Priority Data
Jul. 17, 2020 (CN) 2020106946239

(51) Int. Cl. A61K 35/747; A61P 1/00

(52) U.S. Cl. CPC A61K 35/747; A61P 1/00

(57) ABSTRACT

A strain of Lactobacillus paracasei L.p R3-10 and its application in preparation of medicine for treating ulcerative colitis are disclosed. The Lactobacillus paracasei R3-10 of the present disclosure is a Lactobacillus induced by a low nutritional gradient tolerance, it is obtained after L.p R3 has been domesticated by starvation for 10 generations, and its preservation number is CGMCC No. 19520. The L.p R3-10 of the present disclosure can be prepared as a medicine for treating ulcerative colitis.

### TECHNICAL FIELD

The present disclosure relates to the biotechnology field, and more specifically, to a strain of Lactobacillus paracasei L.p R3-10 and its application in preparation of medicines for treating ulcerative colitis.

### BACKGROUND

Ulcerative colitis (UC) is a chronic non-specific intestinal inflammation whose etiology and pathogenesis are not yet fully clear. The main symptoms are recurrently diarrhea, pus and blood in the stool, abdominal pain, and tenesmus. In recent years, with the improvement of people's living standards and changes in diet, the incidence of this disease has increased year by year. It is generally believed that the pathogenesis of UC may involve the interaction of genetics, microorganisms, the body's immune system, and environmental factors, and its treatment lacks specificity. At present, the commonly used drugs for clinical treatment of UC are aminosalicylic acid preparations, glucocorticoids and immunosuppressants. But these three drugs can only temporarily control and relieve clinical symptoms by inhibiting inflammation and immune response. It also has side effects such as long-term medication, large side effects, easy recurrence after stopping the medication, and the clinical application is limited. Therefore, it is imperative to find safe and effective new preventive drugs. Probiotics are a class of active microorganisms that are beneficial to the host. When ingested in sufficient quantities, they can colonize the host and maintain the balance of the host's intestinal flora, thereby they can exert beneficial effects. At present, the microorganisms that can be used as probiotics are mainly lactic acid bacteria, which are roughly divided into three categories: lactobacillus, Bifidobacterium and gram-positive cocci. Probiotics have gone through many years of exploration and development from discovery to clinical application, and they have begun to enter the public's field of vision. The clinical applications of its related products are also becoming more and more extensive. The functions mainly include regulating gastrointestinal disorders, enhancing intestinal immune function, anti-allergic reactions and protecting the cardiovascular system. The use of probiotics to prevent and treat many gastrointestinal diseases including irritable bowel syndrome (IBS) and inflammatory bowel has become a research hotspot. Many studies worldwide have confirmed that probiotics have good prospects in the prevention and treatment of intestinal diseases such as UC, but their effects are strain-specific.

Therefore, providing a strain of Lactobacillus paracasei and its application in the preparation of medicine for treating ulcerative colitis is an urgent problem for those skilled in the art.

### SUMMARY

The present disclosure provides a strain of Lactobacillus paracasei R3-10 (L.p R3-10) and its application in preparation of medicines for treating ulcerative colitis.

In order to achieve the above objectives, the present disclosure adopts the following technical solutions:

A strain of L.p R3-10, and its preservation number is CGMCC No. 19520, which has been preserved in China General Microbiological Culture Collection Center, referred to as CGMCC, and its address is at Institute of Microbiology, Chinese Academy of Sciences, No. 3 of No. 1 Beichen West Road, Chaoyang District, Beijing. The preservation date is 2020 On March 30. It was classified and named as Lactobacillus paracasei.

Further, the application of the L.p R3-10 in the preparation of medicines for treating ulcerative colitis.

It can be known from the above technical solutions that, compared with the prior art, the present disclosure provides a strain of L.p R3-10 and its application in the preparation of medicines for treating ulcerative colitis. L.p R3-10 is a Lactobacillus paracasei induced by low nutritional gradient tolerance. Lactobacillus paracasei R3 (L.p R3) was derived from infant feces in our laboratory, and L.p R3-10 is obtained after L.p R3 was acclimated by starvation for 10 generations; L.p R3-10 can be prepared as a medicine for treating ulcerative colitis.

### BRIEF DESCRIPTION OF THE DRAWINGS

In order to explain the embodiments of the present invention or the technical solutions in the prior art more clearly, the following drawings that need to be used in the description of the embodiments or the prior art will be briefly introduced. Obviously, the drawings in the following description are only embodiments of the present invention. For those of ordinary skill in the art, other drawings can be obtained based on the drawings disclosed without creative work.

FIG. 1 is the colony morphology of L.p R3-10 of the present disclosure on a MRS agar plates.

FIG. 2 is the colony morphology of L.p R3-10 of the present disclosure on an anaerobic blood agar plate.

FIG. 3 is the morphology of L.p R3-10 of the present disclosure under a Gram stain microscope, 1000 times lens.

FIG. 4 is the percentage change in body weight of the mouse model of ulcerative colitis of the present disclosure.

FIG. 5 is the DAI score of the mouse model of ulcerative colitis of the present disclosure.

FIG. 6 is the colon morphology of each group of mice of the present disclosure; wherein, A is the NS group, B is the DSS group, C is the MSLZ group, D is the L.p R3-10 group, and E is the L.p R3-10+MSLZ group.

FIG. 7 is the colon length of the mouse model of ulcerative colitis of the present disclosure; wherein, *, P<0.5; **, P<0.1.

FIG. 8 is the HE staining result of colon tissue in the mouse model of ulcerative colitis of the present disclosure; wherein, A is the NS group, B is the DSS group, C is the MSLZ group, D is the L.p R3-10 group, and E is the L.p R3-10+MSLZ group.

FIG. 9 is histological damage scores of mice in each group of the present disclosure.

### DETAILED DESCRIPTION OF EMBODIMENTS

Certain exemplary embodiments will now be described to provide an overall understanding of the principles of the structure, function, manufacture, and use of the instruments and methods disclosed herein. One or more examples of these embodiments are illustrated in the accompanying drawings. Those skilled in the art will understand that the instruments and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

### Embodiment 1 Isolation, Culture and Identification of the Precursor Strain L.p R3 of L.p R3-10

### (1) Sample Source

Healthy babies aged 0-6 months were selected as volunteers from the families of school staff. Two weeks before sampling, a normal diet is required and there is no recent history of intestinal infection or antibiotic use. The morning stool is collected on the day of sampling. After collection, the intelligent microbial separation system of Nanjing FMT Medical Co. Ltd is used to separate fecal bacteria. After the separation was completed, the crude fecal bacteria liquid was quickly collected, and the cryopreservation protection solution was added. Then it was placed in an ultra-low temperature refrigerator at −80° C. for later use.

### (2) Isolation, Culture and Identification of L.p R3

1 mL of crude fecal bacteria solution was taken and added to 9 mL of normal saline, and mixed well and performed gradient dilution. 100 μL of each of the bacterial solution with a dilution concentration of 10-5˜10-7 was pipetted, and was intensively spread on MRS medium, BBL medium, M17 medium and anaerobic blood plate, and was cultured at 37° C. for 48 h-72 h under anaerobic conditions. Preliminarily according to the colony characteristics and Gram staining microscopic examination, single colonies were selected for pure culture on the corresponding agar medium. After pure cultured, an appropriate number of bacterial cells was placed in the strain preservation tube and stored in the refrigerator at −80° C. for later use. After the pure strain was transferred to the MRS agar plate for 2 generations, a single colony was selected for Gram stain microscopy and catalase test, and the gram-positive bacilli with typical morphological characteristics of lactobacilli and negative catalase test were initially identified Lactobacillus.

### (3) Biochemical Identification and Sequencing Identification of L.p R3

The above-mentioned Lactobacillus was sent to Dongguan Meikang Biotechnology Co., Ltd. for strain identification. The company uses TIANamp Bacteria DNA Kit (Tiangen, Beijing) to extract DNA from bacteria. Through bacterial universal primer 27F/1492 R, the bacterial 16S rDNA fragment close to the full length was obtained by PCR expansion, and the DNA sequence of the bacterial 16S rDNA fragment was obtained by DNA sequencing, and the sequence was compared with the existing DNA in GenBank and RDP databases. The sequence is compared and analyzed to obtain the species information with the most similar sequence, and the species information of the identified microorganism is inferred based on the sequence similarity. It was identified that the number YSJ03 strain was Lactobacillus paracasei, named L.p R3.

### Embodiment 2 Induction and Identification of L.p R3-10

### (1) Inducing L.p R3 to Become L.p R3-10 by Low Nutritional Gradient Tolerance Method

The frozen L.p R3 was taken out of the −80° C. refrigerator and put into a 37° C. warm water bath to quickly thaw. The thawed bacterial solution was poured into an anaerobic blood agar plate, and placed under anaerobic conditions at 37° C. for 48 hours. The growth of the colonies in the plate and the formation of hemolytic loops were observed. The morphology of the strains under the Gram staining microscope was observed. After no contamination was confirmed, the colonies were transferred to MRS agar plates and cultured at 37° C. under anaerobic conditions for 24 hours. A single colony on the plate was picked and inoculated in 6 mL of MRS liquid medium, cultured and activated under anaerobic conditions at 37° C. for 16-18 h. The activated bacterial solution was inoculated into 100 mL of MRS broth at 3% (v/v) inoculum size, and cultured with shaking at 37° C. for 16-18 h at 120 rpm/min. After centrifugation at 3500 rpm/min for 10 min, the supernatant was discarded, and the cells were harvested after resuspension and washing twice in PBS (pH 7.2-7.4). With PBS, the concentration of the bacterial solution was adjusted to 2 McFarland standard turbidity and was divided into sterile 2 mL EP tubes. Each tube was divided into 1 mL bacterial suspension, divided into 15 tubes, and cultured at 37° C. with shaking. Starting from 0 h, 1 EP tube was taken out every 12 h, and 100 μL of bacterial solution was sucked, spread on the surface of MRS medium, and cultured at 37° C. for 48 h under anaerobic conditions. The single colony with the longest survival time was picked, and the same process was performed again. After 10 generations of circulation, the L.p R3-10 strain induced by low nutritional gradient tolerance was obtained.

### (2) L.p R3-10 Culture Characteristics

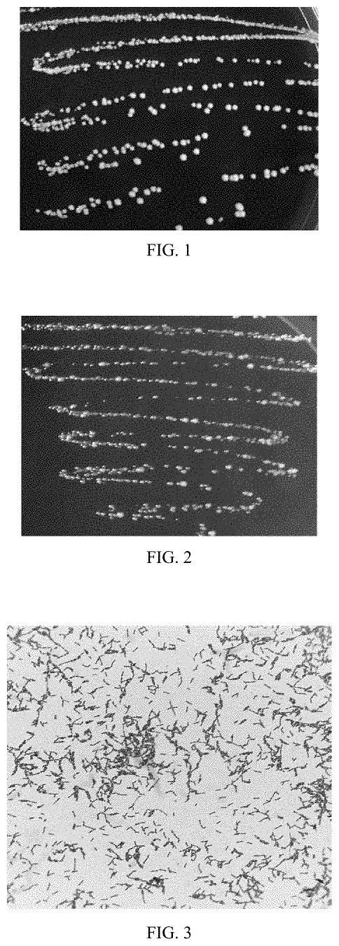

The colonies of L.p R3-10 were picked with an inoculating loop, and inoculated on MRS agar plates and anaerobic blood agar plates in sections and streaks. MRS agar plates were cultured at 37° C. aerobic conditions for 48 h, and anaerobic blood agar plates were cultured at 37° C. anaerobic conditions for 48 h; the growth of colonies on the MRS plate and the formation of hemolytic loops on the anaerobic blood agar plate were observed, the results are shown in FIG. 1 and FIG. 2. The results show that L.p R3-10 is facultative anaerobe, and on the MRS plate the colonies are round, medium-sized, raised, slightly white, moist, with neat edges, and are not hemolytic on the anaerobic blood agar plate.

### (3) Gram Staining L.p R3-10 to Observe the Cell Morphology

The L.p R3-10 colony on the MRS agar plate was picked, the smear was Gram stained, and the morphology of the bacterial cells was observed under the oil microscope. The result is shown in FIG. 3. The results showed that L.p R3-10 was a Gram stain-positive bacillus, without spores, without capsule, arranged in short chains.

### (4) Biochemical Identification of L.p R3-10

The API 50CHL bacterial biochemical identification system of French Merieux Biotech was adopted for identification. First, according to the API 50CHL identification reagent strip instructions, the activated L.p R3-10 bacterial suspension is adjusted to 2 McFarland standard turbidity, and added to the 50 micro-biochemical wells on the reagent strip, and the biochemical wells were covered with sterile liquid paraffin. The bacterial suspension was static cultured at 35° C. for 24 hours to observe the results once, then was cultured continually to 48 hours to observe the results again. The result judged: the color of tube 25 changed from purple to black to be positive, and the color of other tubes changed from purple to yellow to be positive, otherwise it was negative. The reaction results of the strains were analyzed with API identification software to obtain the identification results of the strains. The biochemical reaction results of L.p R3-10 are shown in Table 1. The identification result was Lactobacillus paracasei subsp. casein 1, with an identification rate of 99.7% and a T value of 0.79.

TABLE 1
Biochemical reaction results of L.p R3-10

| | 0 | 1 | 2 | 3 | 4 | 5 | 6 | 7 | 8 | 9 | 10 | 11 | 12 |
|---|---|---|---|---|---|---|---|---|---|---|---|---|---|
| 24 h | − | − | − | − | − | + | − | − | − | − | + | + | + |
| 48 h | − | − | − | − | − | + | − | − | − | − | + | + | + |
| | 0 | GLY | ERY | DARA | LARA | RIB | DXYL | LXYL | ADO | MDX | GAL | GLU | FRU |

| | 13 | 14 | 15 | 16 | 17 | 18 | 19 | 20 | 21 | 22 | 23 | 24 | 25 |
|---|---|---|---|---|---|---|---|---|---|---|---|---|---|
| 24 h | + | + | − | − | − | + | − | − | − | + | − | − | + |
| 48 h | + | + | − | − | − | + | + | − | − | + | − | + | + |
| | MNE | SBE | RHA | DUL | INO | MAN | SOR | MDM | MDG | NAG | AMY | ARB | ESC |

| | 26 | 27 | 28 | 29 | 30 | 31 | 32 | 33 | 34 | 35 | 36 | 37 | 38 |
|---|---|---|---|---|---|---|---|---|---|---|---|---|---|
| 24 h | + | + | − | + | − | + | + | − | + | − | − | − | − |
| 48 h | + | + | + | + | − | + | + | − | + | − | − | − | − |
| | SAL | CEL | MAL | LAC | MEL | SAC | TRE | INU | MLZ | RAF | AMD | GLYG | XLT |

| | 39 | 40 | 41 | 42 | 43 | 44 | 45 | 46 | 47 | 48 | 49 |
|---|---|---|---|---|---|---|---|---|---|---|---|
| 24 h | − | + | − | + | − | − | − | − | + | − | − |
| 48 h | − | + | − | + | − | − | − | − | + | − | − |
| | GEN | TUR | LYX | TAG | DFUC | LFUC | DARL | LARL | GNT | 2KG | 5KG |

Wherein, No. 0 tube was a blank control tube.

### Embodiment 3 Establishment of a Mouse Model of Ulcerative Colitis

25 SPF female C57BL/6 mice (6-8 weeks old, body weight 16-18 g) were selected. The mice were randomly divided into 5 groups: normal group (normal saline, NS group), model group (DSS group), mesalazine group (MSLZ group), L.p R3-10 group (109 CFU/mL), L.p R3-10 combined with mesalazine group (L.p R3-10+MSLZ group), 5 rats in each group. Before modeling, the normal group and the model group were given an equal volume of normal saline. The other three groups were treated by gavage for 7 days with mesalazine (52 mg/mL), L.p R3-10 bacterial solution (1×109 CFU/mL), L.p R3-10 bacterial liquids (1×109 CFU/mL) combined with mesalazine (52 mg/mL). At the beginning of modeling, the normal group was free to drink double distilled water, and the remaining 4 groups of mice were free to drink 3% DSS for 7 days of acute UC modeling. At the same time, they were treated by gavage with sterile normal saline, mesalazine (52 mg/mL), L.p R3-10 bacterial solution (1×109 CFU/mL), L.p R3-10 bacterial solution (1×109CFU/mL) combined with mesalazine (52 mg/mL), 0.2 ml each time, once a day. On the 8th day of modeling, the mice were sacrificed by neck-breaking in the morning, the colons were taken out, and part of the distal colon tissues were fixed in formaldehyde, embedded in paraffin, sectioned, and stained with HE. During the experiment, the weight, stool characteristics and occult blood of the mice were recorded every day.

### Embodiment 4 Evaluation of Mouse Models of Ulcerative Colitis

### (1) General Situation Assessment

The general conditions of each group of mice's eating, activity, hair, etc. were observed daily, and the disease activity index (DAI) score (Table 2) was performed to evaluate the degree of colitis disease activity. Every morning, the weight of each mouse was weighed with an electronic balance and feces were collected to calculate the percentage of weight loss.

Percentage of weight loss=(body weight on day 0−body weight on day n)/body weight on day 0×100%. According to the standards in the table below, the mice were scored and the experimental results were recorded.

According to the experimental animal's weight loss percentage, stool viscosity (normal, loose stools, watery stools) and stool occult blood (normal, occult blood positive, occult blood strong positive), a comprehensive score was made. The total score of the three results was divided by 3 to get the colitis DAI value, that is, the colitis DAI=(weight loss percentage score+stool morphology score+stool occult blood score)/3.

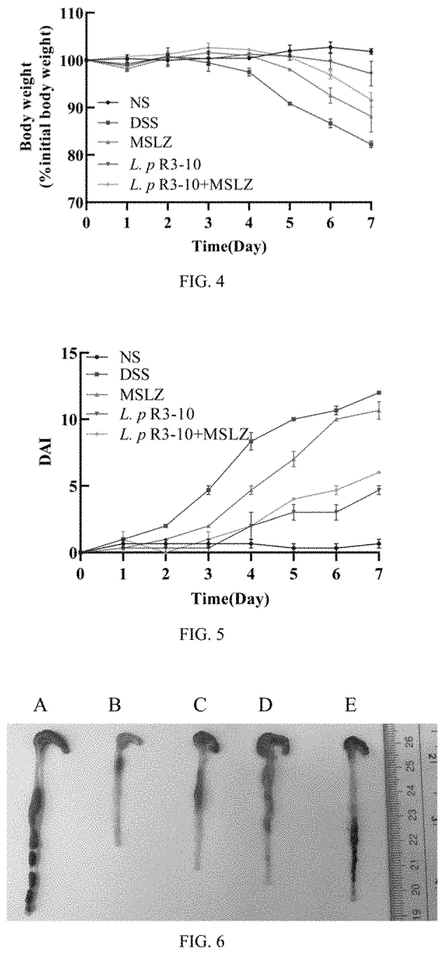

The results of the 7-day body weight percentages of the experimental mice in each group are shown in FIG. 4. The results show that the NS group remained basically unchanged, and the DSS group showed a significant decline from the 4th day, and the 7th day body weight percentage was about 84%; the body weight of MSLZ group, L.p R3-10+MSLZ group and L.p R3-10 group began to decrease from the 5th day, and the degree of decrease gradually decreased.

The DAI results of experimental mouse colitis in each group are shown in FIG. 5. The results show: DSS group>MSLZ group>L.p R3-10+MSLZ group>L.p R3-10 group>NS group.

TABLE 2
Disease activity index scoring standards

| | Percentage of weight | | |
|---|---|---|---|
| Score | loss | Stool morphology | Stool occult blood |
| 0 | unchanged | Normal | Negative (−) |
| 1 | 1-5% | | |
| 2 | 6-10% | Loose stools | Positive (+) |
| 3 | 11-15% | | |
| 4 | >15% | Watery stools | Strong positive (++) |

### (2) Colon Length

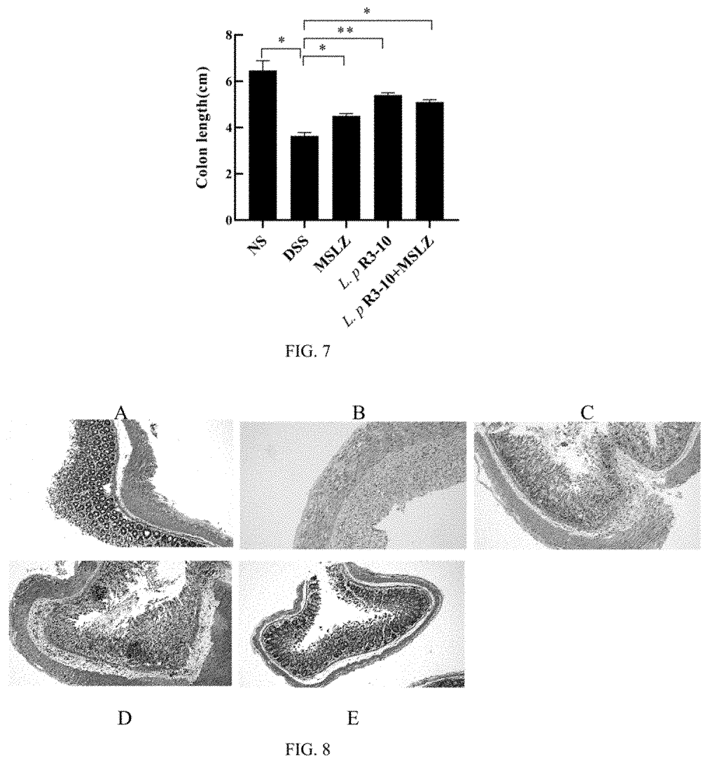

The animals of each group were killed by cervical dislocation method, and the colons were taken out. The morphology of the colon of each group of mice is shown in FIG. 6. The colon length of each group of mice was measured with a ruler, the changes of colon length after dissection of the mice were measured, the average values and standard deviations of each group were calculated, and the relevant statistical analysis was performed. The results are shown in FIG. 7. The results in FIG. 7 show that the average colon length of mice in each group is: NS group>L.p R3-10 group>L.p R3-10+MSLZ group>MSLZ group>DSS group.

### (3) HE Staining of Mouse Colon Tissue

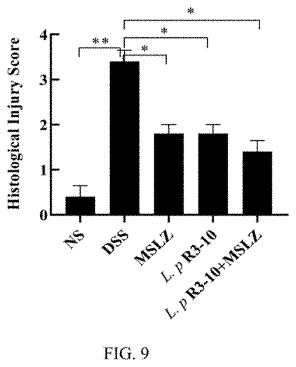

\textcircled{1} After the mice were sacrificed, the colons of the mice in each group were taken out, fixed with 10% formaldehyde solution, embedded in paraffin and sectioned. \textcircled{2} The paraffin sections were immersed in the xylene solution and heated in a microwave oven for 5 min; again, the paraffin sections were immersed in the xylene solution and heated in the microwave oven for 5 min; then they were immersed in absolute ethanol, 95% ethanol, 85% ethanol, and 75% ethanol solution, for 1 min, twice; then rinsed with tap water. \textcircled{3} The paraffin sections were stained with hematoxylin for 5 min, washed with running water, differentiated with 1% hydrochloric acid alcohol, and rinsed again with running water; stained with eosin for 1 min. They were again immersed in 75% ethanol, 85% ethanol, 95% ethanol, and anhydrous ethanol solution, for 1 min, twice, and then immersed in xylene solution, for 5 min, twice, and then sealed with neutral resin. \textcircled{4} Observing and taking pictures under a microscope. Observation and comparison of pathological changes in the colon of mice in each group, such as mucosal epithelial cell changes, crypt structure and texture, inflammatory cell infiltration, were performed under an optical microscope. The results are shown in FIG. 8. The results in FIG. 8 shows that the NS group had a normal structure; DSS group showed fewer mucosal epithelial cells, disordered crypt structure and texture, and inflammatory cell infiltration, which was manifested as UC; the histopathologies of MSLZ group, L.p R3-10 group and L.p R3-10+MSLZ group were obviously improved than that of DSS group.

### (4) Evaluation of Histological Damage

Histological damage score: 0 points, normal and no inflammatory cell infiltration; 1 point, slight inflammatory cell infiltration, no damage to the submucosal tissue; 2 points, moderate inflammatory cell infiltration and submucosal tissue damage (the damage range is 10%˜25%); 3 points, obvious inflammatory cell infiltration, destruction of submucosal tissues, and thickening of the colon wall (the damage range is 25% to 50%); 4 points, severe inflammatory cell infiltration, large-scale colon tissue damage (damage scope>50%) and thickening of colon wall. The evaluation results are shown in FIG. 9. The results show that the colon tissue damage scores of mice in the L.p R3-10 group and the L.p R3-10+MSLZ group were significantly lower than those in the DSS group.

Described above are merely illustrative of the disclosure to enable those skilled in the art to implement or use the disclosure, and are not intended to limit the invention. It should be understood that any modifications, replacements or changes made by those skilled in the art without departing from the spirit of the disclosure should fall within the scope of the disclosure.

1. A strain of Lactobacillus paracasei L.p R3-10, wherein a preservation number of the strain of Lactobacillus paracasei L.p R3-10 is CGMCC No. 19520.

2. An application of the Lactobacillus paracasei L.p R3-10 of claim 1 in preparation of a medicine for treating ulcerative colitis.
